(19) United States
(12) Patent Application Publication
(10) Pub. No.: US 2024/0189483 A1
(43) Pub. Date: Jun. 13, 2024

(54) 3D-BIOPRINTED SCAFFOLDS FOR TISSUE REGENERATION

(71) Applicant: Allegro 3D, Inc., San Diego, CA (US)

(72) Inventors: Wei Zhu, San Diego, CA (US); Wei Wei, San Diego, CA (US)

(21) Appl. No.: 17/755,432

(22) PCT Filed: Oct. 30, 2020
(86) PCT No.: PCT/US2020/058244

§ 371 Date: Apr. 28, 2022

Related U.S. Application Data
(60) Provisional application No. 62/929,724, filed on Nov. 1, 2019.

(51) Int. Cl. A61L 27/38; A61L 27/52; A61L 27/54; B33Y 50/00; B33Y 70/00; B33Y 80/00

(52) U.S. Cl. CPC A61L 27/3878; A61L 27/3834; A61L 27/52; A61L 27/54; B33Y 80/00; A61L 2300/414; A61L 2400/12; A61L 2430/32; B33Y 50/00; B33Y 70/00

(57) ABSTRACT

Disclosed are systems, methods, and devices for tissue regenerative implants. In some aspects, a nerve tissue regeneration implant article includes an exterior shell; a plurality of fascicle structures disposed in an interior region of the exterior shell, where each fascicle structure includes a hollow region between a proximal end and a distal end, such that a fascicle structure is configured to facilitate and guide axonal growth along at least a portion of the fascicle structure between the proximal end and the distal end; and a plurality of vascularizable passages along the exterior shell, wherein the vascularizable passages are configured to allow vascular tissue to infiltrate the implant article, such that the implant article is able to facilitate nerve regeneration by enabling exchange of nutrients, oxygen, and/or waste to axons within the plurality of fascicle structures via the vascular tissue that infiltrates the implant article through the plurality of vascularizable passages.

### CROSS-REFERENCE TO RELATED APPLICATIONS

This patent document claims priorities to and benefits of U.S. Provisional Patent Application No. 62/929,724, titled “3D-BIOPRINTED SCAFFOLDS FOR TISSUE REGENERATION” and filed on Nov. 1, 2019. The entire content of the aforementioned patent application is incorporated by reference as part of the disclosure of this patent document.

### TECHNICAL FIELD

This patent document relates to tissue constructs or scaffolds for various biomedical applications.

### BACKGROUND

Tissue regeneration can involve the use of a tissue scaffold for the formation of new viable tissue for a medical purpose.

### SUMMARY

Disclosed are systems, methods, articles and compositions for tissue regenerative implants with functional elements such as cells, nanoparticles, and/or biomolecule or structures embedded therein during their fabrication, such as 3D bioprinting.

Existing 3D bioprinting techniques are only capable of producing 3D-printed scaffolds formed with the polymer only (i.e., no cells), requiring cells to be manually filled into the channels after 3D printing. Employing a system and method in accordance with embodiments of the disclosed technology, one or multiple types of cells could be embedded in the scaffolds directly with a defined heterogenous distribution using 3D bioprinting (to mimic the cell composition, such as of native spinal cord tissue). Thus, cells are not only located inside the channels, but also embedded in the walls of the channels and the core of the scaffolds. In some embodiments, a physiologically-informed implant device is three-dimensionally printed to include cells embedded inside for therapeutic applications for assisting in repair of injured nerves in a patient, including central nervous system (e.g., the spinal cord) and peripheral nerves (e.g., sciatic or facial nerves). In some embodiments in accordance with the present technology, the three-dimensionally printed implant device does not include the cells embedded therein.

In some embodiments in accordance with the present technology, a biocompatible implant article for nerve regeneration includes an exterior shell; a plurality of fascicle structures disposed in an interior region within the exterior shell, wherein the plurality of fascicle structures each include a hollow region between a proximal end and a distal end, such that a fascicle structure is configured to facilitate and guide axonal growth along at least a portion of the fascicle structure between the proximal end and the distal end; and a plurality of vascularizable passages along the exterior shell, wherein the vascularizable passages are configured to allow vascular tissue to infiltrate the implant article, such that the implant article is able to facilitate nerve regeneration by enabling exchange of nutrients, oxygen, and/or waste to axons within the plurality of fascicle structures via the vascular tissue that infiltrates the implant article through the plurality of vascularizable passages.

In some embodiments in accordance with the present technology, a biocompatible implant article for nerve regeneration includes a plurality of fascicle structures each including one or more walls that surround a hollow region between two openings positioned at a proximal end and a distal end of a fascicle structure, wherein the plurality of fascicle structures are positioned in an interior of the implant article and facilitate and guide axonal growth by allowing axons of nerve cells to enter the hollow region of a fascicle structure and contact the one or more walls of the fascicle structure to grow in a direction between the two openings; and a plurality of channels formed by apertures on an outer perimeter of the implant article that pass through at least some of the fascicle structures disposed within the interior of the implant article, wherein the plurality of channels are configured to allow vascular tissue to infiltrate the implant article, such that the implant article is able to facilitate nerve tissue regeneration by enabling exchange of nutrients, oxygen, and/or waste to the axons of the nerve cells within the plurality of fascicle structures via the vascular tissue that infiltrates the implant article through the plurality of channels.

In some embodiments in accordance with the present technology, a method for repairing transected nerve injuries includes (a) implanting an implant article on at least one severed end of a transected nerve, wherein the implant article comprises an exterior shell, a plurality of fascicle structures in an interior region within the exterior shell, and a plurality of vascularizable passages to allow host vascular networks to infiltrate the implant article that can facilitate nerve regeneration by enabling exchange of nutrients, oxygen, and/or waste; and (b) repairing the transected nerve by facilitating growth of nerve fibers from at least one severed end of a transected nerve to at least one severed end of another transected nerve to bridge a gap between the transected nerve.

The subject matter described in this patent document can be implemented in specific ways that provide one or more of the following features.

### BRIEF DESCRIPTION OF THE DRAWINGS

FIGS. 1A and 1B show diagrams of an example embodiment of a non-functionalized physiologically informed implant in accordance with the disclosed technology showing hexagonal hollow fascicle microstructures.

FIGS. 2A and 2B show diagrams of an example embodiment of a functionalized physiologically informed implant in accordance with the disclosed technology showing hexagonal hollow fascicle microstructures.

FIGS. 3A and 3B show diagrams of an example embodiment of a heterogeneous non-functionalized physiologically informed implant in accordance with the disclosed technology showing hexagonal hollow fascicle microstructures, representing the white matter region along with an interior butterfly shaped area representing the gray matter region of the central nervous system.

FIGS. 4A and 4B show diagrams of an example embodiment of a heterogeneous functionalized physiologically informed implant in accordance with the disclosed technology showing hexagonal hollow fascicle microstructures, representing the white matter region along with an interior butterfly shaped area representing the gray matter region of the central nervous system.

FIGS. 5A and 5B show diagrams of an example embodiment of a heterogeneous non-functionalized physiologically informed implant in accordance with the disclosed technology showing hexagonal hollow ridged fascicle microstructures, representing the white matter region along with an interior butterfly shaped area representing the gray matter region of the central nervous system.

FIGS. 6A and 6B show diagrams of an example embodiment of a heterogeneous functionalized physiologically informed implant in accordance with the disclosed technology showing hexagonal hollow ridged fascicle microstructures, representing the white matter region along with an interior butterfly shaped area representing the gray matter region of the central nervous system.

FIGS. 7A and 7B show diagrams of an example embodiment of a heterogeneous functionalized physiologically informed implant in accordance with the disclosed technology showing hexagonal fascicle microstructures with defined regional stiffness representing the white matter region along with an interior butterfly shaped area representing the gray matter region of the central nervous system.

FIG. 8 shows panels of images depicting example 3D-printed scaffolds that fill various spinal cord lesion morphologies and etiologies at human scale.

FIG. 9 shows panels of images and diagrams depicting various example nerve implant designs, including example 3D printed human life-size nerve implants.

FIG. 10 shows panels of intraoperative photographs of example nerve implants at the surgical nerve repair site taken before implantation, right after implantation and 2 weeks after implantation.

### DETAILED DESCRIPTION

Currently, repairing nerve injuries, especially spinal cord injuries (SCI), remain as one of the most challenging tasks in the clinical field. According to the recent report released by the National Spinal Cord Injury Statistical Center (NSCISC) in 2019, there are approximately 291,000 SCI patients in the U.S. with 17,730 new cases each year, resulting in substantial psychological and economic costs to both patients and caregivers. Due to the hostile microenvironment created by SCI, spontaneous regeneration of axons in the injured adults is lacking. Currently there are no clinically approved therapies to promote axonal regeneration and recovery of function after SCI.

Tissue engineering methods have emerged as promising treatment strategies for SCI. Conventional techniques include grafting a variety of cells with matrix materials to lesion sites to provide a more favorable environment for axonal regeneration. However, these cellular grafts do not provide any guidance for the directional axonal growth, resulting in random axon orientations that reduces the probability for optimal functional recovery. Templated scaffolds with linear channels have also been developed to precisely guide the axons to grow linearly across the lesion sites. Although the number of axons reaching across the lesion to the appropriate distal targets increased significantly with the guidance of the linear channels, there existed reactive cell layer (RCL) and extracellular matrix (ECM) containing collagen that encapsulated the scaffolds preventing the regenerating axons form exiting the scaffold channels and reaching the distal host spinal cord. This is a common issue with in vivo implants due to the poor biocompatibility of the scaffolding materials. More importantly, the templating method lacks the flexibility and capability to customize implants that can fit perfectly in the lesion site of the SCI patients. Thus, there remains a critical need for the physiologically informed implants that can provide optimal guidance and assistance to the axonal regeneration and functional recovery of the SCI patients.

In addition to SCI, peripheral nerve injuries require approximately 200,000 surgeries annually. Injuries result from trauma, tumors, and other illnesses, and may cause complete or partial paralysis. Current repair strategies for peripheral nervous system (PNS) injury after a complete nerve transection involve suturing the distal and proximal nerve ends without introducing tension or placing an autologous nerve graft (autograft) harvested from some other part of the body to treat larger defects. The autograft is the current “gold standard” but requires additional procedures to harvest the graft, and often leads to neuroma formation and loss of function at the donor site. Furthermore, the total length of the autologous donor grafts is limited, especially in children, thereby limiting reconstructive options.

The fundamental goal of tissue engineering is to create materials that can replace or repair injured tissues. To that end it is desirable to have tissue engineered constructs that mimic the architecture of native tissues. However, current technology is insufficient at recreating complex architectures that are 3D, span multiple length scales, have interconnected pores and features, and are constructed from natural biopolymers.

Disclosed are systems, methods, articles and compositions for tissue regenerative structural constructs that are implantable in living tissue, which can be 3D bioprinted, and which can include functional elements embedded therein during printing. The disclosed structural constructs are referred to as “scaffolds,” “implants,” or “physiologically informed implants” as described in this patent document.

The disclosed technology can be implemented in ways that address the above and other technical challenges by providing physiologically-informed implants that are designed to significantly improve functional recovery of the nerve injury patients. In some embodiments, the disclosed technology includes a system and a method for (1) providing an implant with the shape and dimension matching that of the lesion site perfectly; (2) providing vascularizable passages that can allow for the penetration of host vasculature into the implants for transportation of oxygen, nutrients, and waste; (3) providing fascicle structures with physical and mechanical guidance to facilitate axonal regeneration; (4) using biocompatible materials with minimal foreign body reaction and reduced reactive cell layer that can deflect axonal growth; and/or (5) directly embedding functional elements, such as cells, nanoparticles, growth factors and/or other biomolecules, in the implants with biomimetic, localized and organized distribution that function to reconnect the disconnected proximal and distal nerve ends at the lesion site. Implementations of the disclosed technology can provide an effective and patient-specific therapy for the patients with nerve injuries such as peripheral nerve injuries and spinal cord injuries for which there are still no clinically approved cures.

In some embodiments, for example, a device includes physiologically informed implants articles (with or without cells embedded inside) that can be implemented for a variety of purposes, including therapeutic purposes to repair the injured nerves in patients, such as in central nervous system (e.g., the spinal cord) and peripheral nervous system (e.g., sciatic or facial nerves). In some embodiments, one or more of the following five features or aspects are included in the devices, methods and systems in accordance with the disclosed technology.

### I. Physiologically Informed Designs

Modern imaging technologies such as magnetic resonance imaging (MRI) and computed tomography (CT) scans can be used to determine the shape and dimension of the lesion site in the patients. Rapid prototyping (e.g., 3D printing and bioprinting) and other biofabrication techniques can be used to manufacture the physiologically informed implants that match the lesion site perfectly.

### II. Fascicle Structures

Axons in the white matter of healthy native spinal cord are highly organized in fascicles to transmit information and signals from the rostral end to the caudal end. Thus, to mimic such organization, the example implant articles and devices can include engineered biomaterial structures, referred to as “fascicle structures” or “fascicle microstructures”, that provide guidance to the axonal growth and regeneration so that the signal way from the proximal to the distal end of the lesion site can be reconnected. The engineered fascicle structures are designed using biocompatible materials to form a wall or walls that surround a hollow interior between two openings, e.g., sometimes referred to as the proximal and distal (open) ends of the fascicle structure. The wall or walls of the fascicle structures can be configured in different geometries such that the axons of nerve cells can enter through at least one of the open ends and contact the interior side of the wall or walls within the hollow interior region of the fascicle structure and grow in a direction between the two openings. For example, without the fascicle structures, the regenerating axons may grow in random directions without enough of axons reaching to the distal end, thus limiting the functional recovery.

### III. Vascularizable Passages

The example implant articles and devices can include micropores as vascularizable passages on the perimeter shell of the implants to allow and facilitate the penetration of the host vasculature into the implants to provide the necessary transportation of oxygen, nutrients, and waste. The vascularizable passages are openings configured on an exterior (e.g., perimeter shell) of the implant. In some embodiments, the vascularizable passages include a patterned or non-patterned array or arrangement of openings on the exterior of the implant and through at least some of the fascicle structures disposed within the implant. In some embodiments, the vascularizable passages have an arrangement of openings on the exterior of the implant and through at least some of the fascicle structures disposed within the implant. For example, the arrangement of vascularizable passages through the fascicle structures can be produced via 3D printing techniques of the fascicle structures. The vascularizable structures act to sustain the viability of the cells inside the implants and achieve functional recovery in the long term.

### IV. Biocompatible Materials

Biocompatible materials can be used for constructing the implants. In some embodiments, for example, the biocompatible materials include hydrogels (e.g., gelatin, polyethylene glycol, collagen, hyaluronic acid, alginate, and their derivatives conjugated with acrylate or methacrylate groups), elastomers [e.g., poly(glycerol sebacate) and its derivatives conjugated with acrylate groups], and conductive polymers [e.g., polypyrrole, polyaniline, PEDOT:PSS or poly(3,4-ethylenedioxythiophene) polystyrene sulfonate]. For example, the selected polymer materials are biocompatible such that the implant will induce minimal foreign body reaction and reduce the reactive cell layer that can impede the regeneration of axons into the fascicle structures. They are also biodegradable so that the biomaterials can be resorbed by the host with the regeneration of the nerve.

### V. Functional Elements

In some embodiments, to achieve optimal nerve regeneration, functional elements such as cells, growth factors, nanoparticles, nanocomposites, and biomolecules, can be embedded into the fascicle structures to further assist the regeneration of the injured nerves. The said cell types include but are not limited to induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). In one embodiment, iPSC-derived neural cells can form synapses with the host axons and function to reconnect the previously disconnected proximal and distal ends in the lesion site.

Various examples of embodiments and implementations of the physiologically informed implant articles, devices, systems and methods in accordance with the disclosed technology are described below.

### EXAMPLE EMBODIMENTS AND IMPLEMENTATIONS

### Example 1: Non-Functionalized Physiologically Informed Implants+Fascicles+Vascularizable Passages

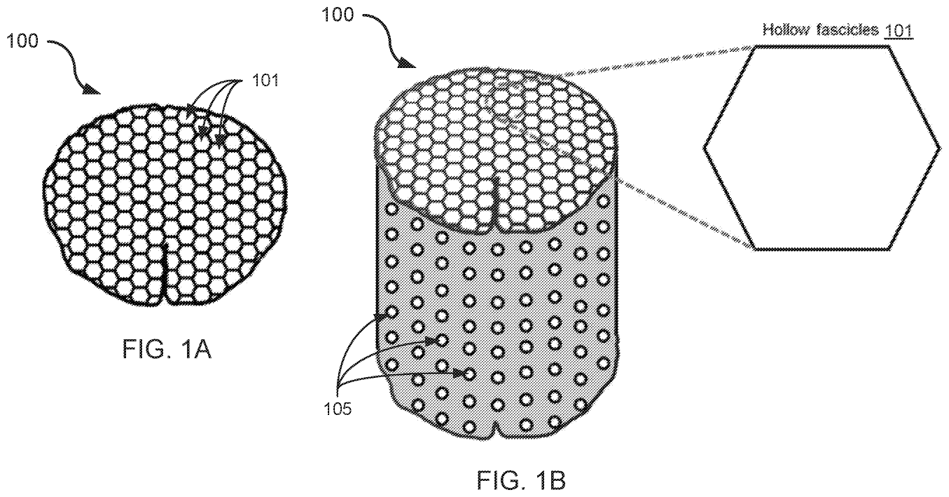

FIGS. 1A and 1B show diagrams of an example embodiment of a non-functionalized (e.g., acellular) physiologically informed implant article, labeled 100, having hollow fascicle microstructures 101 (top view in FIG. 1A), with a side view (in FIG. 1B) showing vascularizable passages on the perimeter shell of the example acellular physiologically informed implant 100, with zoomed-in cross-section view illustrating a hexagonal geometry of each hollow fascicle microstructures 101.

The structure and size of the implant article 100, e.g., including the hollow fascicle microstructures, can be designed using data from magnetic resonance imaging (MRI) and computed tomography (CT) scans to produce patient-specific and personalized implant structures that perfectly match the lesion site. For example, manufacturing methods to produce the example implants can include rapid prototyping techniques among other biofabrication methods. For instance, 3D printing technologies such as digital light processing (DLP)-based 3D printers can be employed to fabricate said implants due to their superior resolution, fabrication speed, and ability to accommodate building highly complex geometries. Biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene sulfonate).

In some particular embodiments, the biomaterial selection criteria for producing the implants can include: 1) mechanical stiffness matched to host tissue to minimize inflammatory responses and reduce the reactive cell layer that can impede the regeneration of host axons, 2) biocompatibility to reduce foreign body immune reactions and enhance regenerative cascades such that host axonal cells are able to grow into the fascicles, 3) biodegradability such that the implant can be replaced by host tissues over time, and/or 4) mechanically compliant to handling during surgery and suturing. In some embodiments, for example, the mechanical stiffness of the implant article that is matched to the host tissue can be configured to be between about 10 kilopascals (kPa) to about 100,000 kPa. Some example ranges of mechanical stiffness matched to the host tissue for various implementations include: about 100 kPa to about 500 kPa, about 200 kPa to about 600 kPa, about 1,000 kPa to about 1,500 kPa, about 10 kPa to about 10,000 kPa, about 10,000 kPa to about 100,000 kPa, about 200 kPa to about 100,000 kPa, about 50 kPa to about 200 kPa, about 2,000 kPa to about 6,000 kPa, or about 100 kPa to about 700 kPa.

As shown in FIG. 1A, the example acellular physiologically informed implant article 100 includes fascicle structures 101 in the implant interior are designed to facilitate and guide host axonal growth from the proximal to distal ends. In the example acellular physiologically informed implant article 100 shown in FIG. 1A, the fascicle structures 101 have a hexagonal structure. However, the fascicle structures can be any hollow structure that can facilitate nerve growth. For example, the disclosed fascicle structures can be configured in other shapes, including but not limited to triangular, square, rectangular, pentagonal, hexagonal, heptagonal, octagonal, ellipse, or trapezoidal geometries, for various tissue engineering applications in accordance with the present technology. In some embodiments, the fascicle structures 101 are configured to have a diameter of about 0.01 μm to about 1000 μm. Some examples of the diameter range of the fascicle structures 101 include: about 0.01 μm to about 10 μm, about 0.01 μm to about 100 μm, about 1 μm to about 10 μm, about 1 μm to 100 μm, about 10 μm to 100 μm, or about 10 μm to 1000 μm.

Furthermore, as depicted in FIG. 1B, the example acellular physiologically informed implant article 100 includes patterned vascularizable passages 105 along the exterior shell of the implant to allow host vascular networks to infiltrate the implant and improve nerve regeneration by enabling the exchange of nutrients, oxygen, and waste. In some embodiments, the vascularizable passages 105 are configured to have a diameter between about 0.01 μm to about 1000 μm. For example, the vascularizable passages 105 can have a diameter in a range between about 0.01 μm to about 10 μm, about 0.01 μm to about 100 μm, about 1 μm to about 10 μm, about 1 μm to 100 μm, about 10 μm to 100 μm, or about 10 μm to 1000 μm.

### Example 2: Functionalized Physiologically Informed Implants+Fascicles+Vascularizable Passages

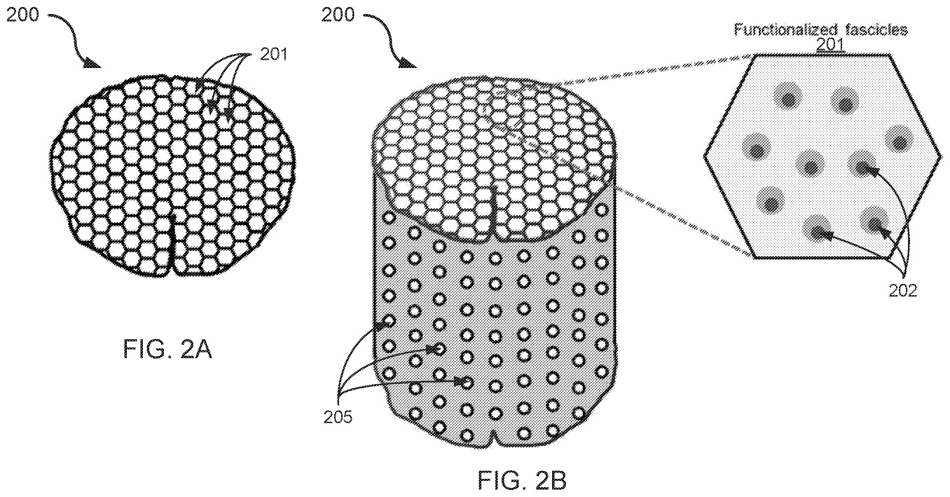

FIGS. 2A and 2B show diagrams of an example embodiment of a functionalized physiologically informed implant article, labeled 200, showing hollow fascicle microstructures 201 (top view in FIG. 2A), with a side view (in FIG. 2B) showing vascularizable passages on the perimeter shell of the example functionalized physiologically informed implant 200, with zoomed-in cross-section view illustrating a hexagonal geometry of each functionalized fascicle 201. In the example embodiment shown in FIGS. 2A and 2B, the hollow fascicle microstructures 201 have a hexagonal structure.

The example embodiment of the implant article 200 shown in FIGS. 2A and 2B is a derivative of the example in FIGS. 1A-1B that includes functionalized fascicles. For example, the example functionalized physiologically informed implant 200 includes functional elements 202, e.g., including but not limited to cells, growth factors, nanoparticles, and other biomolecules, which can be embedded within the fascicle structures 201 to further assist the regeneration of injured nerves. For instance, the presence of cells can help further regenerative activities by synapsing with host axons to form complete nerve connections throughout the implant construct. Examples of cell types that can be incorporated include, but are not limited to, induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). Examples of growth factors include, but are not limited to, neurotrophic factors such as brain-derived neurotrophic factor (BDNF), vascular endothelial growth factor (VEGF), basic fibroblast growth factor (bFGF), calpain inhibitor MDL28170, and glial cell-derived neurotrophic factor (GDNF). Examples of nanoparticles to improve electrical conduction and neurite growth include, but are not limited to, piezoelectric properties of boron nitride nanotubes, gold nanorods, carbon nanotubes, and graphene. Such nanoparticles can also be mixed with polymers to form conductive nanocomposites for constructing the nerve implants and further assisting the axon regeneration and connection. Examples of polymers include, but are not limited to, biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene sulfonate).

### Example 3: Heterogeneous Non-Functionalized Physiologically Informed Implants+Fascicles+Vascularizable Passages

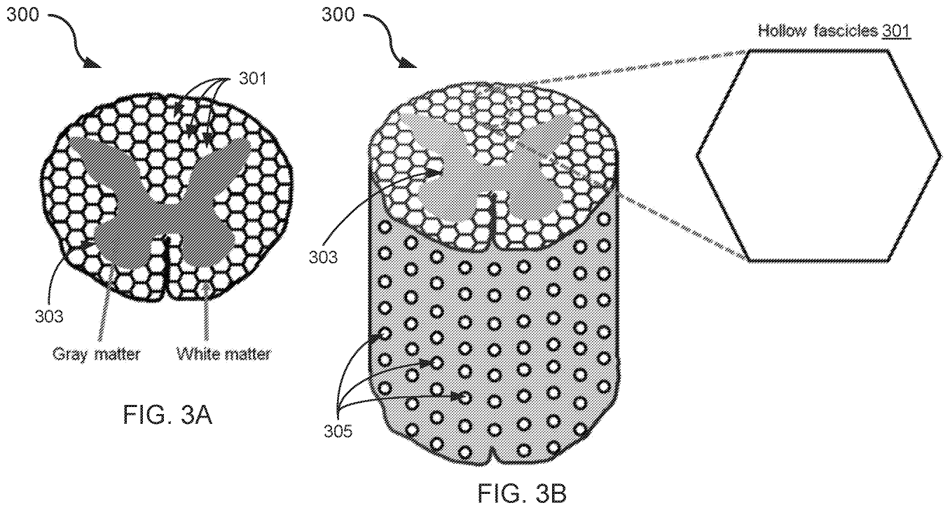

FIGS. 3A and 3B show diagrams of an example embodiment of a heterogeneous non-functionalized (e.g., acellular) physiologically informed implant article, labeled 300, showing hollow fascicle microstructures 301 representing the white matter region along with an interior butterfly shaped area 303 representing the gray matter region of the central nervous system. In the example embodiment shown in FIGS. 3A and 3B, the hollow fascicle microstructures 301 have a hexagonal structure. FIG. 3A shows a top view of the example heterogeneous non-functionalized physiologically informed implant 300. FIG. 3B shows a side view of the example heterogeneous acellular physiologically informed implant 300 depicting vascularizable passages 305 on the perimeter shell. Also shown in FIG. 3B, is a zoomed-in cross-section view illustrating the hexagonal geometry of each hollow fascicle 301. In this example, the gray matter region can also include designed microstructures prepared from biomaterials different from the white matter region. For example, an implant article, such as the example implant 300, having a grey matter region and white matter region comprised of different biomaterials affects the heterogeneity of the implant article 300 and allows it to more accurately mimic the biological environment in which the implant article 300 is embedded.

The example embodiment of the implant article 300 shown in FIGS. 3A and 3B is a derivative of the example in FIGS. 1A-1B that includes heterogenous regions representative of the white and gray matter within the central nervous system. In this example, the design of the implant 300 can be used to realize more complete regenerative functionalities critical for nerve repair since white matter functions to transmit nerve impulses between neurons while the gray matter functions to control muscular and sensory activities. In this embodiment, the heterogenous white and gray matter design also serves to create a more biomimetic implant that can be used to enhance nerve regeneration and improve mechanical integrity of the implant through the incorporation of other said biomaterials. For instance, the gray matter can be prepared from a different biomaterial than the surrounding white matter region along with incorporation of internal microstructures (e.g., hexagonal fascicles, solid, etc.). When the gray and white matter regions are comprised of different biological materials, each region can have a different biological function and thus, the implant article 300 can be used to mimic that heterogenicity of a biological environment.

### Example 4: Heterogeneous Functionalized Physiologically Informed Implants+Fascicles+Vascularizable Passages

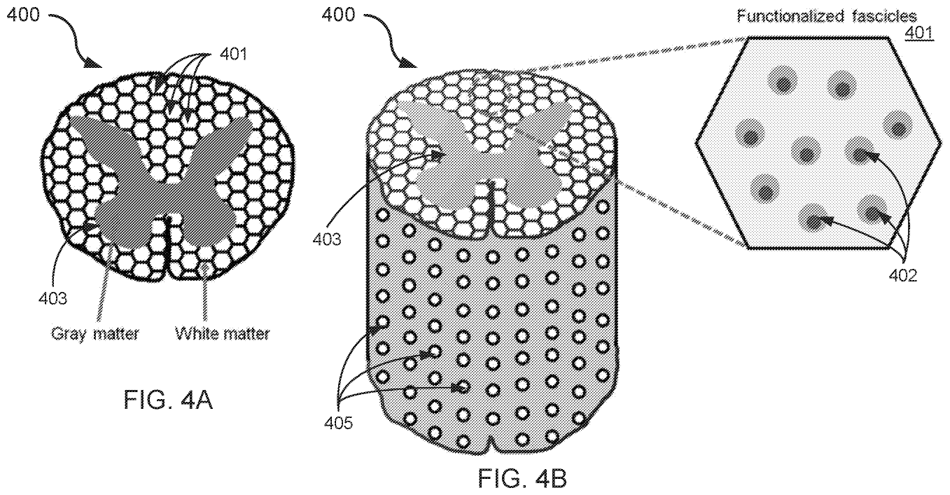

FIGS. 4A and 4B show diagrams of an example embodiment of a heterogeneous functionalized physiologically informed implant article, labeled 400, showing hollow fascicle microstructures 401 representing the white matter region along with an interior butterfly shaped area 403 representing the gray matter region of the central nervous system. In the example embodiment shown in FIGS. 4A and 4B, the hollow fascicle microstructures 401 have a hexagonal structure. FIG. 4A shows a top view of the example heterogeneous functionalized physiologically informed implant 400. FIG. 4B shows a side view of the example heterogeneous physiologically informed implant 400 depicting vascularizable passages 405 on the perimeter shell. Also shown in FIG. 4B is a zoomed-in cross-section view illustrating the hexagonal geometry of each hollow fascicle 401. In this example, the gray matter region can also be functionalized and include designed fascicle microstructures 401 prepared from biomaterials different from the white matter region.

The example embodiment of the implant article 400 shown in FIGS. 4A and 4B is a derivative of the example in FIGS. 3A-3B that includes functionalization of the implant 400 to recapitulate the biochemically distinct and differing cell populations within the white and gray matter regions of the central nervous system. As illustrated in FIG. 4B, the implant article 400 includes functional elements 402, including but not limited to cells, growth factors, nanoparticles, and other biomolecules, which can be embedded within the fascicle structures to further assist the regeneration of injured nerves. For instance, the presence of cells can help further regenerative activities by synapsing with host axons to form complete nerve connections throughout the implant construct. Examples of cell types that can be incorporated include but are not limited to induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). Examples of growth factors include but are not limited to neurotrophic factors such as brain-derived neurotrophic factor (BDNF), vascular endothelial growth factor (VEGF), basic fibroblast growth factor (bFGF), calpain inhibitor MDL28170, and glial cell-derived neurotrophic factor (GDNF). Examples of nanoparticles to improve electrical conduction and neurite growth include but are not limited to conductive and/or piezoelectric properties of boron nitride nanotubes, gold nanorods, carbon nanotubes, and graphene. Such nanoparticles can also be mixed with polymers to form conductive nanocomposites for constructing the nerve implants and further assisting the axon regeneration and connection. Examples of polymers include, but are not limited to, biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene.

### Example 5: Heterogeneous Non-Functionalized Physiologically Informed Implants+Ridged Fascicles+Vascularizable Passages

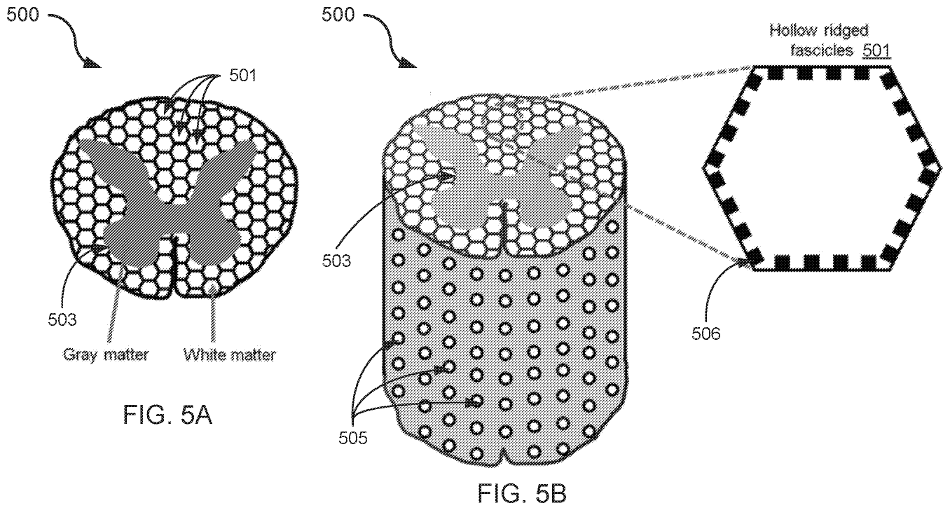

FIGS. 5A and 5B show diagrams of an example embodiment of a heterogeneous non-functionalized (e.g., acellular) physiologically informed implant article, labeled 500, showing hollow ridged fascicle microstructures 501 representing the white matter region along with an interior butterfly shaped area 503 representing the gray matter region of the central nervous system. In the example embodiment shown in FIGS. 5A and 5B, the hollow fascicle microstructures 501 have a hexagonal structure. FIG. 5A shows a top view of the example heterogeneous non-functionalized (e.g., acellular) physiologically informed implant 500. FIG. 5B shows a side view of the example heterogeneous acellular physiologically informed implant 500 showing vascularizable passages 505 on the perimeter shell. Also shown in FIG. 5B is a zoomed-in cross-section view illustrating hexagonal geometry of each hollow ridged fascicle 501. The gray matter region shown can also include designed microstructures prepared from biomaterials different from the white matter region.

The example embodiment of the implant article 500 shown in FIGS. 5A and 5B is a derivative of the example in FIGS. 3A-3B that includes nanostructure or microstructure ridges 506 within the interior surface of each hollow fascicle microstructures 501. The ridges 506 are designed to provide physical guidance cues to further facilitate directional growth of the host axons along the proximal to distal end of the implant article 500. In some implementations of the implant article 500, the ridges 506 are 3D-printed onto the hollow fascicle microstructures 501. In some embodiments, the ridges 506 can be configured to have a thickness between about 0.001 μm to about 500 μm. For example, between about 0.001 μm to about 1 μm, 0.001 μm to about 10 μm, about 0.001 μm to about 100 μm, about 0.001 μm to about 200 μm, about 0.001 μm to about 300 μm, about 0.001 μm to about 400 μm, about 1 μm to about 10 μm, about 1 μm to about 100 μm, about 1 μm to about 200 μm, about 1 μm to about 300 μm, about 1 μm to about 400 μm, about 100 μm to about 200 μm, about 100 μm to about 300 μm, about 100 μm to about 400 μm, or about 100 μm to about 500 μm.

### Example 6: Heterogeneous Functionalized Physiologically Informed Implants+Ridged Fascicles+Vascularizable Passages

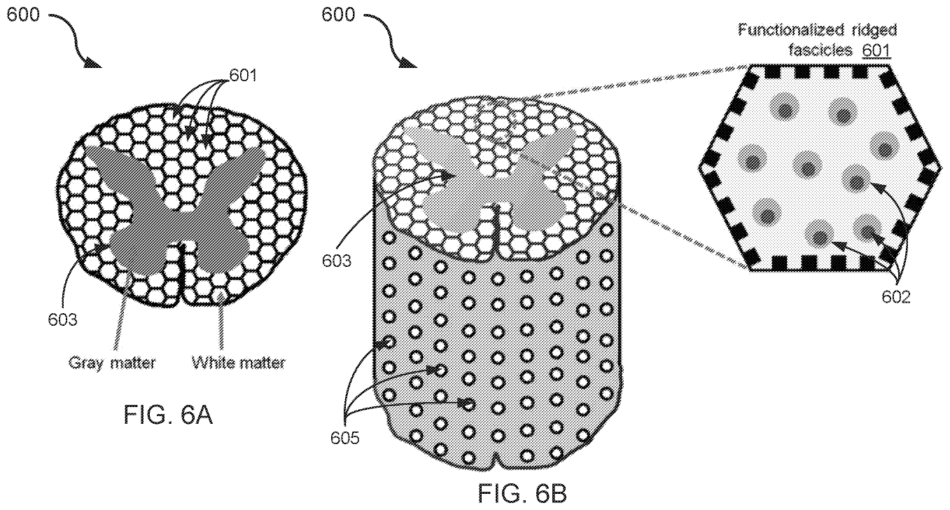

FIGS. 6A and 6B show diagrams of an example embodiment of a heterogeneous functionalized physiologically informed implant article, labeled 600, showing hollow ridged fascicle microstructures 601, representing the white matter region along with an interior butterfly shaped area 603 representing the gray matter region of the central nervous system. In the example embodiment shown in FIGS. 6A and 6B, the hollow fascicle microstructures 601 have a hexagonal structure. FIG. 6A shows a top view of the example heterogeneous functionalized physiologically informed implant 600. FIG. 6B shows a side view of the example heterogeneous functionalized physiologically informed implant 600 showing vascularizable passages 605 on the perimeter shell. Also shown in FIG. 6B is a zoomed-in cross-section view illustrating hexagonal geometry of each functionalized fascicle 601. The gray matter region can also be functionalized and include designed microstructures prepared from biomaterials different from the white matter region.

The example embodiment of the implant article 600 shown in FIGS. 6A and 6B is a derivative of the example in FIGS. 5A-5B that includes functionalization of the implant 600 to recapitulate the biochemically distinct and differing cell populations within the white and gray matter regions of the central nervous system. As shown in FIG. 6B, the example heterogeneous functionalized physiologically informed implant article 600 includes functional elements 602, including but not limited to cells, growth factors, nanoparticles, and other biomolecules, which can be embedded within the fascicle structures to further assist the regeneration of injured nerves. For instance, the presence of cells can help further regenerative activities by synapsing with host axons to form complete nerve connections throughout the implant construct. Examples of cell types that can be incorporated include but are not limited to induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). Examples of growth factors include but are not limited to neurotrophic factors such as brain-derived neurotrophic factor (BDNF), vascular endothelial growth factor (VEGF), basic fibroblast growth factor (bFGF), calpain inhibitor MDL28170, and glial cell-derived neurotrophic factor (GDNF). Examples of nanoparticles to improve electrical conduction and neurite growth include but are not limited to conductive and/or piezoelectric properties of boron nitride nanotubes, gold nanorods, carbon nanotubes, and graphene. Such nanoparticles can also be mixed with polymers to form conductive nanocomposites for constructing the nerve implants and further assisting the axon regeneration and connection. Examples of polymers include, but are not limited to, biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene.

### Example 7: Heterogeneous Functionalized Physiologically Informed Implants+Defined Regional Stiffness of Fascicles+Vascularizable Passages

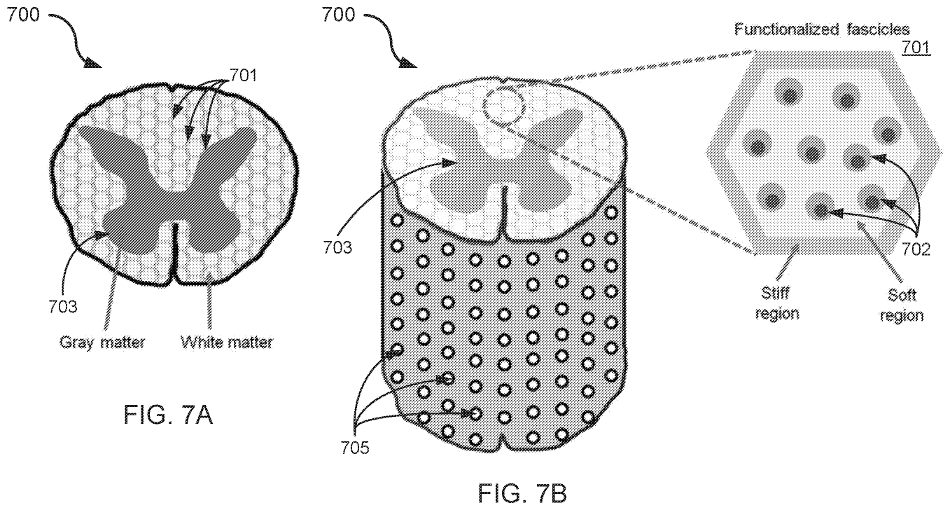

FIGS. 7A and 7B show diagrams of an example embodiment of a heterogeneous functionalized physiologically informed implant article, labeled 700, showing ridged fascicle microstructures 701 representing the white matter region along with an interior butterfly shaped area 703 representing the gray matter region of the central nervous system. In the example embodiment shown in FIGS. 7A and 7B, the hollow fascicle microstructures 701 have a hexagonal structure. FIG. 7A shows a top view of the example heterogeneous functionalized physiologically informed implant 700. FIG. 7B shows a side view of the example heterogeneous functionalized physiologically informed implant 700 showing vascularizable passages 705 on the perimeter shell.

Also shown in FIG. 7B is a zoomed-in cross-section view illustrating hexagonal geometry of each functionalized fascicle 701 formed using grayscale DLP-based 3D printing to incorporate localized stiff regions and soft regions as physical guidance cues. In some embodiments, the stiff and soft regions are made of the same biomaterials but are classified as “stiff” or “soft” based on the polymer crosslinking density that affects the stiffness or the softness of the material in the regions. The designation of a region as “soft” or “stiff” is relative to the density of adjacent regions. For example, a first region having a density of 100 kPa is “soft” if adjacent to a second region having a density 1000 kPa, where the second region would then be considered “stiff.” However, the first region having a density of 100 kPa is considered “stiff” if adjacent to a second region having a density of 10 kPa, wherein the second region, in this scenario, would be considered “soft.” In some embodiments, the soft region has a density of about 10 kPa, about 100 kPa, and about 1000 kPa, where the density of the soft region is less than the density of an adjacent region. In some embodiments, the stiff region has a density of about 10 kPa, about 100 kPa, and about 1000 kPa, where the density of the stiff region is greater than the density of an adjacent region. In some embodiments, the 3D printing process used to incorporate the localized stiff and soft regions changes the crosslinking density of the polymers used to make the stiff and soft regions. Cells then grow along the interface between stiff and soft regions thereby imparting directionality (e.g., along the interface of the stiff and soft regions) to cell growth. The gray matter region can also be functionalized and include designed microstructures prepared from biomaterials different from the white matter region.

The example embodiment of the implant article 700 shown in FIGS. 7A and 7B is a derivative of the example in FIGS. 4A-4B that includes the defined regional stiffness within each fascicle and functionalization of the said implant to recapitulate the biochemically distinct and differing cell populations within the white and gray matter regions of the central nervous system. As shown in FIG. 7B, the defined regional stiffness of the hollow fascicle microstructures 701, pertaining to the stiff perimeter and soft interior, can be fabricated using a grayscale DLP-based 3D printing technique. For example, specifically, grayscale light projection enables direct control over local light intensities within the same projected digital pattern such that regions receiving a higher light intensity result in a stiffer region compared to a lower light intensity that results in a softer region. These distinct stiff/soft regions provide physical mechanical guidance cues to facilitate directional growth of embedded cells and host axons from the proximal to distal end of the implant. In some implementations in accordance with the example embodiment of the implant article 700, for example, the cells grow along to the interface between the stiff and soft regions.

As shown in FIG. 7B, the example heterogeneous functionalized physiologically informed implant article 700 includes functional elements 702, including but not limited to cells, growth factors, nanoparticles, and other biomolecules, which can be embedded within the fascicle structures to further assist the regeneration of injured nerves. For instance, the presence of cells can help further regenerative activities by synapsing with host axons to form complete nerve connections throughout the implant construct. In the example heterogeneous functionalized physiologically informed implant article 700 shown in FIG. 7B, the functional elements 702 represent cells having a nucleus (inner circle) surrounded by a cell wall (outer circle). Examples of cell types that can be incorporated include but are not limited to induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). Examples of growth factors include but are not limited to neurotrophic factors such as brain-derived neurotrophic factor (BDNF), vascular endothelial growth factor (VEGF), basic fibroblast growth factor (bFGF), calpain inhibitor MDL28170, and glial cell-derived neurotrophic factor (GDNF). Examples of nanoparticles to improve electrical conduction and neurite growth include but are not limited to conductive and/or piezoelectric properties of boron nitride nanotubes, gold nanorods, carbon nanotubes, and graphene. Such nanoparticles can also be mixed with polymers to form conductive nanocomposites for constructing the nerve implants and further assisting the axon regeneration and connection. Examples of polymers include, but are not limited to, biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene.

### Example Application: Spinal Cord Injury Repair

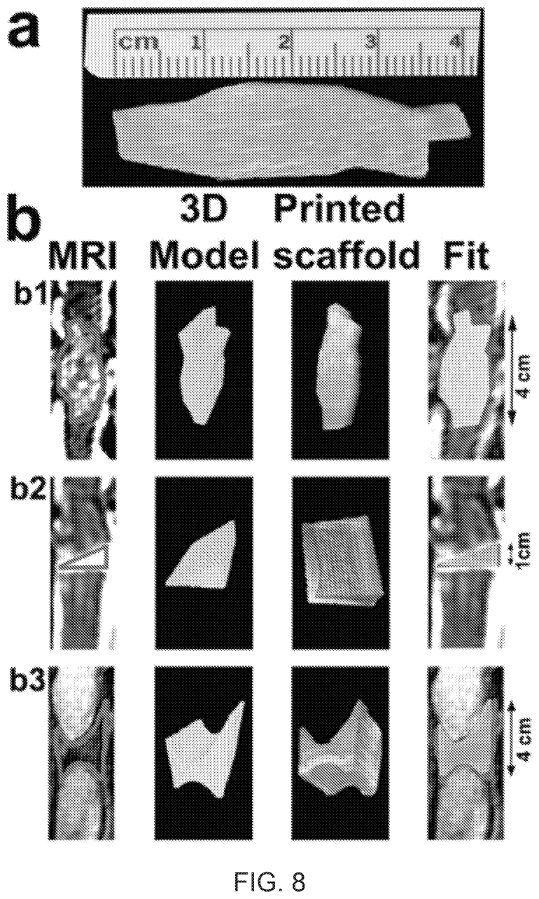

One application of the example embodiments of the disclosed implant articles in this patent document is to treat spinal cord injuries, for which there are currently no clinically approved therapies to promote axonal regeneration and recovery of function. Combined with 3D bioprinting, patient specific implants can be rapidly printed with different sizes and irregular shapes to conform to individual patient lesion sites that can be identified on magnetic resonance imaging (MRI). An example of 3D bioprinted implants conforming the morphology of various human injury cavities is illustrated in FIG. 8.

FIG. 8 shows panels of images depicting example 3D-printed scaffolds that fill various spinal cord lesion morphologies and etiologies at human scale. Panel (a) of FIG. 8 shows a longitudinal view of scaffold printed to 4-cm-long lesion (shown also in b1). Panel (b) of FIG. 8 shows scaffolds printed to various lesion morphologies based on MRI, including (b1,3) irregularly shaped central contusion lesion, and (b2) knife-cut lesion. In FIG. 8 panel (b), the left column indicates sagittal mid-cervical MRI image (b1-2: T2 weighted, b3: T1 weighted); lesion site is indicated by red line. In FIG. 8 panel (b), the second-from-left column indicates a 3D model was developed corresponding to the lesion shape. In FIG. 8 panel (b), the 3rd column-from-left indicates 3D-printed scaffold. In FIG. 8 panel (b), the right column shows overlay of printed scaffold design in lesion cavity.

### Example Application: Peripheral Nerve Repair

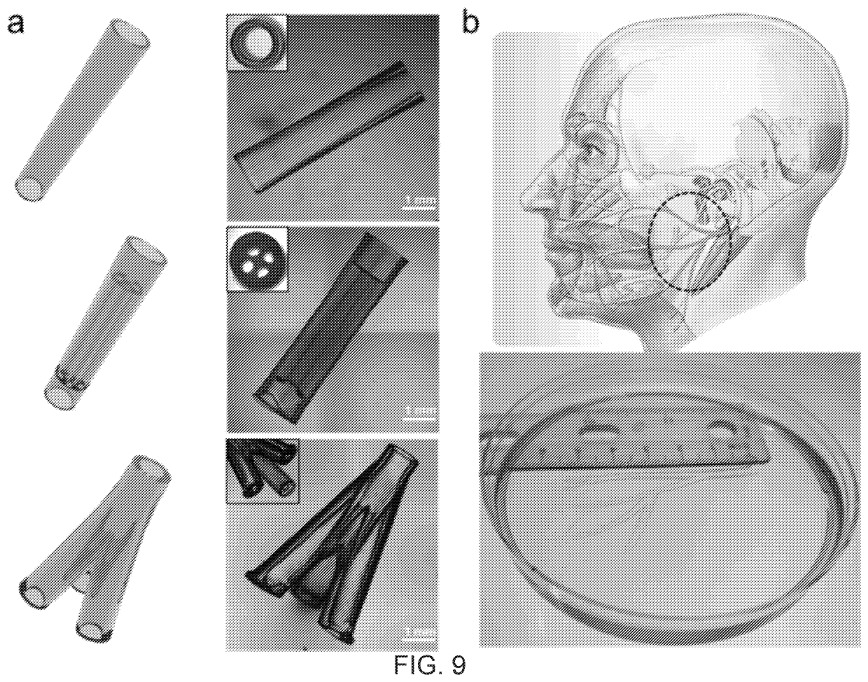

Another exemplary application of the disclosed technology is to repair peripheral nerve injuries which sometimes require branched nerve implants. Combined with 3D bioprinting, for example, various shapes of nerve implants can be rapidly printed to connect the injured nerves (as shown in FIG. 9), such as a simple hollow conduit, a conduit with multiple microchannels, a branched conduit, and an anatomically appropriate sized-biomimetic conduit for human facial nerve repair.

FIG. 9 shows panels of images and diagrams depicting various example nerve implant designs (left column of panel (a)) and the corresponding 3D printed implants (right column of panel (a)), including example 3D printed human life-size nerve implants (shown in bottom image of panel (b), illustrated in diagram of panel (b)).

As demonstrated by example data shown in FIG. 9, the CAD design of the nerve implants are shown on the left column and the 3D printed nerve implants are shown on the right column. The complex branched human facial nerve structure is recapitulated with an anatomically appropriate sized nerve implant (e.g., 5.5 cm in length) featuring the zygomatic branches, the buccal branches, the marginal mandibular branch, and the cervical branch (FIG. 9b). In some embodiments, the size of the implant varies based on the size of the injury and/or treatment needs. It is believed that such nerve implants with such high complexity and large scale have never been achieved before.

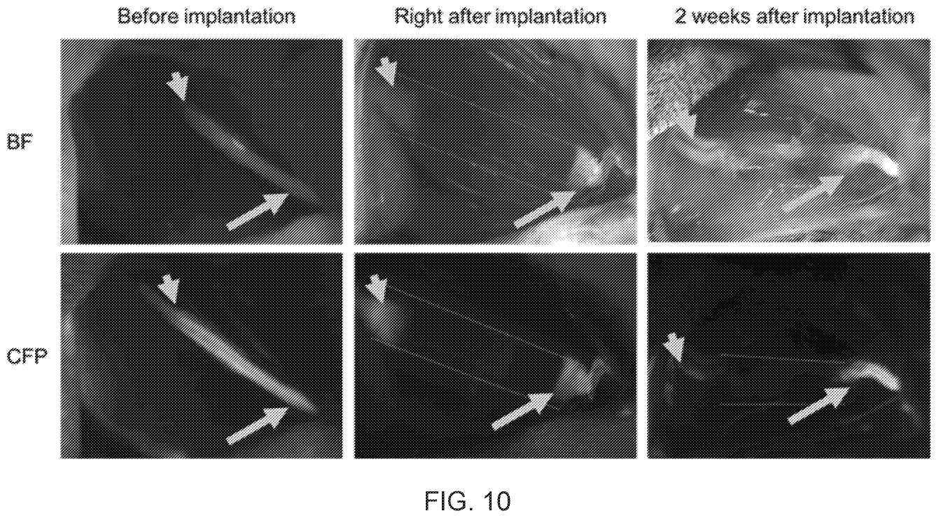

The 3D printed nerve implants can be readily used to guide nerve regeneration in vivo, as demonstrated by example data shown in FIG. 10. In this example implementation, for example, a complete sciatic nerve transection was introduced to the transgenic mice expressing cyan fluorescent protein (CFP) under a neuron specific (Thy-1) promoter. Following the transection, the proximal and distal ends of the nerves retracted to create a nerve gap approximately 4 mm in distance. The 3D-printed nerve implants were then implanted to connect the proximal and distal nerve ends, which were aligned with the microchannels.

FIG. 10 shows panels of intraoperative photographs of example nerve implants (e.g., bright field/CFP fluorescence) at the surgical nerve repair site taken before implantation, right after implantation and 2 weeks after implantation. The example images of FIG. 10 present the process of the surgical implantation in both bright field view and CFP view to highlight the nerve. Two weeks after implantation, the proximal segment of the nerve can be seen to connect through the transected gap within the nerve implant (blue arrow).

### Example Methods for Tissue Engineering

In some embodiments in accordance with the present technology, the disclosed implantable articles and devices are implementable according to the disclosed methods for repairing transected nerve injuries and other tissue engineering procedures. For example, in some embodiments, the methods include implanting an implant article of the present disclosure near at least one severed end of a transected nerve. In some embodiments, the methods further comprise repairing the transected nerve by facilitating growth of nerve fibers from at least one severed end of a transected nerve to at least one severed end of another transected nerve to bridge a gap between the transected nerve.

In some embodiments, the transected nerve comprises peripheral nerves. In some embodiments, the transected nerve is associated with a spinal cord injury. In some embodiments, the transected nerve is a sciatic nerve.

In some embodiments, the methods further comprise determining a size of the transected nerve injury by magnetic resonance imaging (MRI). In some embodiments, the methods comprise using an implant article with a size that matches the size of the transected nerve injury. In some embodiments, the implant article is about 0.1 cm to about 20 cm in length. For example, about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, or about 20 cm.

In some embodiments, the implant article has one or more branches to facilitate the repair of one or more transected nerves. In some embodiments, the implant article as one, two, three, four, five, or more branches. In some embodiments, the implant article as zygomatic branches, buccal branches, marginal mandibular branches, or cervical branches.

In some embodiments, the methods further comprise introducing one or more of cells, growth factors, or nanoparticles into the implant article through the plurality of vascularizable passages to help facilitate the nerve injury repair. Examples of cell types that can be incorporated include but are not limited to induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). Examples of growth factors include but are not limited to neurotrophic factors such as brain-derived neurotrophic factor (BDNF), vascular endothelial growth factor (VEGF), basic fibroblast growth factor (bFGF), calpain inhibitor MDL28170, and glial cell-derived neurotrophic factor (GDNF). Examples of nanoparticles to improve electrical conduction and neurite growth include but are not limited to conductive and/or piezoelectric properties of boron nitride nanotubes, gold nanorods, carbon nanotubes, and graphene. Such nanoparticles can also be mixed with polymers to form conductive nanocomposites for constructing the nerve implants and further assisting the axon regeneration and connection. Examples of polymers include, but are not limited to, biomaterials applicable for fabricating said implants include hydrogels (e.g., gelatin, polyethylene glycol, collagen, hyaluronic acid, alginate, and their derivatives conjugated with acrylate or methacrylate groups), elastomeric materials (e.g., poly(glycerol sebacate), and conductive polymers (e.g., polypyrrole, polyaniline, PEDOT:PSS or poly(3,4-ethylenedioxythiophene) polystyrene.

In some embodiments, the methods of repairing transected nerve injuries can take at least about 1 week, about 5 weeks, about 10 weeks, about 20 weeks, about 30 weeks, about 1 year, about 2 years, or more.

In some embodiments, the methods comprise removing the nerve implant after nerve repair is complete. In some embodiments, the methods comprise leaving the nerve implant in a subject, wherein the nerve implant is biocompatible and biodegradable.

### EXAMPLES

In various example embodiments and implementations in accordance with the present technology, the disclosed implant articles, devices, systems and methods can include fascicle structures to guide the axonal growth and vascularizable passages on the perimeter shell to facilitate vascularization and anastomosis with the host circulation. The overall design (including dimension and shape) of the example implants can be informed by modern imaging techniques such as magnetic resonance imaging (MRI) and computed tomography (CT) scan. The example implants can be built with polymer materials, such as hydrogels (e.g., gelatin, polyethylene glycol, collagen, hyaluronic acid, alginate, and their derivatives conjugated with acrylate or methacrylate groups), elastomers (e.g., poly(glycerol sebacate) and its derivatives conjugated with acrylate groups), and conductive polymers (e.g., polypyrrole, polyaniline, PEDOT:PSS or poly(3,4-ethylenedioxythiophene) polystyrene sulfonate). The selected polymer materials are biocompatible and biodegradable such that the implant will induce minimal foreign body reaction and reduce the reactive cell layer that can impede the regeneration of axons into the fascicle structures. Functional elements, such as cells, growth factors, nanoparticles, nanocomposites (e.g., polymers mixed with functional nanoparticles), and other biomolecules, can be embedded into the fascicle structures to further assist the regeneration of the injured nerves. The cell types can include, but are not limited to, induced pluripotent stem cells (iPSCs), embryonic stem cells, mesenchymal stem cells, cells differentiated from these stem cells (such as iPSC-derived neural progenitor cells, iPSC-derived oligodendrocytes), and other supportive cells (such as endothelial cells, Schwann cells). The example implants can be manufactured with rapid prototyping techniques (e.g., 3D printing and bioprinting) as well as other conventional microfabrication and biofabrication techniques (e.g., molding).

In one embodiment, for example, the fascicle structures can include a bundle of hollow hexagonal microchannels, which are designed to guide the growth of the axons in the injured nerves. The vascularizable passages can include micropores on the perimeter shell that allows the host vasculature to grow into the implant and vascularize the implant. In some embodiments, for example, the fascicle structures can be filled with functional elements such as cells, growth factors, nanoparticle, nanocomposites (e.g., conductive polymers mixed with functional nanoparticles), and other biomolecules that can connect injured nerves or further facilitate the nerve regeneration across the lesion site. Example of functional elements can be filled manually into the fascicle structures after the implant is manufactured or be directly embedded in the fascicle structures during the biofabrication process, e.g., direct bioprinting with cells. In this example, bioprinting can be used to directly pattern or embed functional elements (e.g., cells, growth factors, nanoparticle, nanocomposites, and other biomolecules) in a predefined distribution pattern (e.g., heterogeneous distribution of multiple cell types, gradient distribution of growth factors) to assist and guide the axon regeneration. In some embodiments, for example, microscale ridges are designed on the inner wall of the fascicle channels which can act as a physical guidance to align the regenerating axons to grow from the proximal end of the lesion site to the distal end. In some embodiments, for example, 3D bioprinting, in particular, digital light processing based bioprinting, can be used to modulate local stiffness while fabricating the fascicle structures and provide a mechanical stiffness interface to guide and regenerating axons to grow from the proximal end of the lesion site to the distal end.

In some embodiments in accordance with the present technology (example 1), a biocompatible implant article for nerve regeneration includes an exterior shell; a plurality of fascicle structures disposed in an interior region within the exterior shell, wherein the plurality of fascicle structures each include a hollow region between a proximal end and a distal end, such that a fascicle structure is configured to facilitate and guide axonal growth along at least a portion of the fascicle structure between the proximal end and the distal end; and a plurality of vascularizable passages along the exterior shell, wherein the vascularizable passages are configured to allow vascular tissue to infiltrate the implant article, such that the implant article is able to facilitate nerve regeneration by enabling exchange of nutrients, oxygen, and/or waste to axons within the plurality of fascicle structures via the vascular tissue that infiltrates the implant article through the plurality of vascularizable passages.
Example 2 includes the implant article of any of examples 1-15, wherein each of the plurality of fascicle structures has a shape comprising one of a circular, triangular, rectangular, pentagonal, hexagonal, heptagonal, octagonal, elliptical, or trapezoidal geometry, or an arbitrary geometry.
Example 3 includes the implant article of any of examples 1-15, wherein the exterior shell is structured to include a gray matter region and a white matter region comprised of different biomaterials to mimic the heterogenicity of a biological system.
Example 4 includes the implant article of any of examples 1-15, wherein each of the plurality of fascicle structures have a diameter in a range of 0.01 μm to 1,000 μm.
Example 5 includes the implant article of any of examples 1-15, wherein the vascularizable passages have a diameter in a range of 0.01 μm to 1000 μm.
Example 6 includes the implant article of any of examples 1-15, wherein the implant article is non-functionalized.
Example 7 includes the implant article of any of examples 1-5 or 8-15, wherein the implant article is functionalized by a functional element embedded within at least some of the plurality of fascicle structures.
Example 8 includes the implant article of example 7, wherein the functional element includes one or more of cells, growth factors, nanoparticles, nanocomposites, or other biomolecules, wherein the functional element is able to further assist the regeneration of an injured nerve.
Example 9 includes the implant article of any of examples 1-15, wherein the exterior shell is structured to include an interior butterfly shaped area to allow a gray matter region of a central nervous system of the living thing within the implant article.
Example 10 includes the implant article of any of examples 1-15, wherein the plurality of fascicle structures includes localized stiff and soft regions providing physical guidance cues for nerve growth.
Example 11 includes the implant article of any of examples 1-15, wherein a structure and size of the implant article are designed using data from magnetic resonance imaging (MRI) and computerized tomography (CT) scans to produce a patient-specific and personalized implant structure that perfectly match a lesion site of nerve tissue.
Example 12 includes the implant article of any of examples 1-15, wherein the implant article is producible by 3D printing.
Example 13 includes the implant article of any of examples 1-15, wherein the implant article is producible using biomaterials including one or more of a hydrogel, an elastomeric materials, or a conductive polymers.
Example 14 includes the implant article of any of examples 1-15, wherein the plurality of fascicle structures is hollow and further comprises ridges along an interior of the plurality of fascicle structures to facilitate and guide axonal growth along at least portion of a fascicle structure across a proximal end to a distal end.
Example 15 includes the implant article of any of examples 1-14, wherein the ridges have a thickness between about 0.001 μm to about 500 μm.

In some embodiments in accordance with the present technology (example 16), a biocompatible implant article for nerve regeneration includes a plurality of fascicle structures each including one or more walls that surround a hollow region between two openings positioned at a proximal end and a distal end of a fascicle structure, wherein the plurality of fascicle structures are positioned in an interior of the implant article and facilitate and guide axonal growth by allowing axons of nerve cells to enter the hollow region of a fascicle structure and contact the one or more walls of the fascicle structure to grow in a direction between the two openings; and a plurality of channels formed by apertures on an outer perimeter of the implant article that pass through at least some of the fascicle structures disposed within the interior of the implant article, wherein the plurality of channels are configured to allow vascular tissue to infiltrate the implant article, such that the implant article is able to facilitate nerve tissue regeneration by enabling exchange of nutrients, oxygen, and/or waste to the axons of the nerve cells within the plurality of fascicle structures via the vascular tissue that infiltrates the implant article through the plurality of channels.
Example 17 includes the implant article of any of examples 16-28, wherein each of the plurality of fascicle structures are structured to have a shape comprising one of a circular, triangular, rectangular, pentagonal, hexagonal, heptagonal, octagonal, elliptical, or trapezoidal geometry, or an arbitrary geometry.
Example 18 includes the implant article of any of examples 16-28, wherein each of the plurality of fascicle structures have a diameter in a range of 0.01 μm to 1,000 μm.
Example 19 includes the implant article of any of examples 16-28, wherein the apertures of the plurality of channels have a diameter in a range of 0.01 μm to 1000 μm.
Example 20 includes the implant article of any of examples 16-28, wherein the plurality of fascicle structures comprise ridges along an interior side of the one or more walls of the plurality of fascicle structures to facilitate and guide the axonal growth between the proximal end and the distal end of the fascicle structures.
Example 21 includes the implant article of example 20, wherein the ridges have a thickness between about 0.001 μm to about 500 μm.
Example 22 includes the implant article of any of examples 16-28, further comprising one or more functional element embedded within at least some of the plurality of fascicle structures to further assist the nerve tissue regeneration, wherein the functional element includes one or more of cells, growth factors, nanoparticles, nanocomposite structures, or one or more biomolecules.
Example 23 includes the implant article of any of examples 16-28, wherein the fascicle structures are arranged within the interior of the implant article to form an interior butterfly shaped area that allows a gray matter region of a central nervous system of a living thing to occupy the interior butterfly shaped area the implant article.
Example 24 includes the implant article of any of examples 16-28, wherein the plurality of fascicle structures are structured to have localized stiff regions and soft regions providing physical guidance cues for nerve growth.
Example 25 includes the implant article of example 24, wherein the localized stiff regions and soft regions are created based on a polymer crosslinking density that affects a relative stiffness or softness of material in the regions of the plurality of fascicle structures.
Example 26 includes the implant article of any of examples 16-28, wherein the implant article comprises one or more biomaterials including one or more of a hydrogel, an elastomeric materials, or a conductive polymers.
Example 27 includes the implant article of any of examples 16-28, wherein a structure and size of the implant article are designed using data from magnetic resonance imaging (MRI) and computerized tomography (CT) scans to produce a patient-specific and personalized implant structure that matches a lesion site of the nerve tissue.
Example 28 includes the implant article of any of examples 16-27, wherein the implant article is producible by 3D printing.

In some embodiments in accordance with the present technology (example 29), a method for repairing transected nerve injuries includes (a) implanting an implant article on at least one severed end of a transected nerve, wherein the implant article comprises an exterior shell, a plurality of fascicle structures in an interior region within the exterior shell, the plurality fascicle structures able to facilitate and guide host axonal growth a long at least a portion of a fascicle structure across a proximal end to a distal end, and a plurality of vascularizable passages along the exterior shell, the vascularizable passages able to allow host vascular networks to infiltrate the implant article that can facilitate nerve regeneration by enabling exchange of nutrients, oxygen, and/or waste; and (b) repairing the transected nerve by facilitating growth of nerve fibers from at least one severed end of a transected nerve to at least one severed end of another transected nerve to bridge a gap between the transected nerve.
Example 30 includes the method of any of examples 29-41, wherein the transected nerve comprises peripheral nerves.
Example 31 includes the method of any of examples 29-41, wherein the transected nerve is associated with a spinal cord injury.
Example 32 includes the method of any of examples 29-41, wherein the transected nerve is a sciatic nerve.
Example 33 includes the method of any of examples 29-41, where a size of the implant articles varies based on a size of the transected nerve injuries.
Example 34 includes the method of any of examples 29-41, wherein the implant article has one or more branches to facilitate repair of one or more transected nerve injuries.
Example 35 includes the method of any of examples 29-41, further comprising determining a size of the transected nerve injuries by magnetic resonance imaging (MRI).
Example 36 includes the method of any of examples 29-41, wherein one or more of cells, growth factors, or nanoparticles are introduced into the implant article through the plurality of vascularizable passages.
Example 37 includes the method of any of examples 29-41, wherein the cells are selected from the group consisting of pluripotent stem cells (iPSCs), embryonic stem cells, mesenchymal stem cells, cells differentiated from stem cells, and other supportive cells.
Example 38 includes the method of any of examples 29-41, wherein the cells differentiated from stem cells include iPSC-derived neural progenitor cell and iPSC-derived oligodendrocytes.
Example 39 includes the method of any of examples 29-41, wherein the other supportive cells include endothelial cells and Schwann cells.
Example 40 includes the method of any of examples 29-41, wherein the growth factors are selected from the group consisting of neurotrophic factor (BDNF), vascular endothelial growth factor (VEGF), basic fibroblast growth factor (bFGF), calpain inhibitor MDL28170, and glial cell-derived neurotrophic factor (GDNF).
Example 41 includes the method of any of examples 29-40, wherein the nanoparticles are selected from the group consisting of boron nitride nanotubes, gold nanorods, carbon nanotubes, and graphene.

It is intended that the specification, together with the drawings, be considered exemplary only, where exemplary means an example. As used herein, the singular forms “a,” “an” and “the” are intended to include the plural forms as well, unless the context clearly indicates otherwise. Additionally, the use of “or” is intended to include “and/or.” unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A biocompatible implant article for nerve regeneration, comprising:
an exterior shell;
a plurality of fascicle structures disposed in an interior region within the exterior shell, wherein the plurality of fascicle structures each include a hollow region between a proximal end and a distal end, such that a fascicle structure is configured to facilitate and guide axonal growth along at least a portion of the fascicle structure between the proximal end and the distal end; and
a plurality of vascularizable passages along the exterior shell, wherein the vascularizable passages are configured to allow vascular tissue to infiltrate the implant article, such that the implant article is able to facilitate nerve regeneration by enabling exchange of nutrients, oxygen, and/or waste to axons within the plurality of fascicle structures via the vascular tissue that infiltrates the implant article through the plurality of vascularizable passages.

2. The implant article of claim 1, wherein each of the plurality of fascicle structures has a shape comprising one of a circular, triangular, rectangular, pentagonal, hexagonal, heptagonal, octagonal, elliptical, or trapezoidal geometry, or an arbitrary geometry.

3. The implant article of claim 1, wherein the exterior shell is structured to include a gray matter region and a white matter region comprised of different biomaterials to mimic the heterogenicity of a biological system.

4. The implant article of claim 1, wherein each of the plurality of fascicle structures have a diameter in a range of 0.01 μm to 1,000 μm.

5. The implant article of claim 1, wherein the vascularizable passages have a diameter in a range of 0.01 μm to 1000 μm.

6. The implant article of claim 1,
wherein the implant article is non-functionalized; or
wherein the implant article is functionalized by a functional element embedded within at least some of the plurality of fascicle structures, and wherein the functional element includes one or more of cells, growth factors, nanoparticles, nanocomposites, or other biomolecules, wherein the functional element is able to further assist the regeneration of an injured nerve.

7. (canceled)

8. (canceled)

9. The implant article of claim 1, wherein the exterior shell is structured to include an interior butterfly shaped area to allow a gray matter region of a central nervous system of the living thing within the implant article.

10. The implant article of claim 1, wherein the plurality of fascicle structures includes localized stiff and soft regions providing physical guidance cues for nerve growth.

11. The implant article of claim 1,
wherein a structure and size of the implant article are designed using data from magnetic resonance imaging (MRI) and computerized tomography (CT) scans to produce a patient-specific and personalized implant structure that perfectly match a lesion site of nerve tissue; or
wherein the implant article is producible by 3D printing; or
wherein the implant article is producible using biomaterials including one or more of a hydrogel, an elastomeric materials, or a conductive polymers.

12. (canceled)

13. (canceled)

14. The implant article of claim 1, wherein the plurality of fascicle structures is hollow and further comprises ridges along an interior of the plurality of fascicle structures to facilitate and guide axonal growth along at least portion of a fascicle structure across a proximal end to a distal end.

15. The implant article of claim 14, wherein the ridges have a thickness between about 0.001 μm to about 500 μm.

16. A biocompatible implant article for nerve regeneration, comprising:
a plurality of fascicle structures each including one or more walls that surround a hollow region between two openings positioned at a proximal end and a distal end of a fascicle structure, wherein the plurality of fascicle structures are positioned in an interior of the implant article and facilitate and guide axonal growth by allowing axons of nerve cells to enter the hollow region of a fascicle structure and contact the one or more walls of the fascicle structure to grow in a direction between the two openings; and
a plurality of channels formed by apertures on an outer perimeter of the implant article that pass through at least some of the fascicle structures disposed within the interior of the implant article, wherein the plurality of channels are configured to allow vascular tissue to infiltrate the implant article, such that the implant article is able to facilitate nerve tissue regeneration by enabling exchange of nutrients, oxygen, and/or waste to the axons of the nerve cells within the plurality of fascicle structures via the vascular tissue that infiltrates the implant article through the plurality of channels.

17. The implant article of claim 16, wherein each of the plurality of fascicle structures are structured to have a shape comprising one of a circular, triangular, rectangular, pentagonal, hexagonal, heptagonal, octagonal, elliptical, or trapezoidal geometry, or an arbitrary geometry.

18. The implant article of claim 16, wherein each of the plurality of fascicle structures have a diameter in a range of 0.01 μm to 1,000 μm.

19. The implant article of claim 16, wherein the apertures of the plurality of channels have a diameter in a range of 0.01 μm to 1000 μm.

20. The implant article of claim 16, wherein the plurality of fascicle structures comprise ridges along an interior side of the one or more walls of the plurality of fascicle structures to facilitate and guide the axonal growth between the proximal end and the distal end of the fascicle structures.

21. The implant article of claim 20, wherein the ridges have a thickness between about 0.001 μm to about 500 μm.

22. The implant article of claim 16, further comprising one or more functional element embedded within at least some of the plurality of fascicle structures to further assist the nerve tissue regeneration, wherein the functional element includes one or more of cells, growth factors, nanoparticles, nanocomposite structures, or one or more biomolecules.

23. The implant article of claim 16, wherein the fascicle structures are arranged within the interior of the implant article to form an interior butterfly shaped area that allows a gray matter region of a central nervous system of a living thing to occupy the interior butterfly shaped area the implant article.

24. The implant article of claim 16 wherein the plurality of fascicle structures are structured to have localized stiff regions and soft regions providing physical guidance cues for nerve growth.

25. The implant article of claim 24, wherein the localized stiff regions and soft regions are created based on a polymer crosslinking density that affects a relative stiffness or softness of material in the regions of the plurality of fascicle structures.

26. The implant article of claim 16,
wherein the implant article comprises one or more biomaterials including one or more of a hydrogel, an elastomeric materials, or a conductive polymers; or
wherein the implant article is producible by 3D printing; or
wherein a structure and size of the implant article are designed using data from magnetic resonance imaging (MRI) and computerized tomography (CT) scans to produce a patient-specific and personalized implant structure that matches a lesion site of the nerve tissue.

27. (canceled)

28. (canceled)

29. A method for repairing transected nerve injuries, the method comprising:
(a) implanting an implant article on at least one severed end of a transected nerve, wherein the implant article comprises:
an exterior shell,
a plurality of fascicle structures in an interior region within the exterior shell, the plurality fascicle structures able to facilitate and guide host axonal growth a long at least a portion of a fascicle structure across a proximal end to a distal end, and
a plurality of vascularizable passages along the exterior shell, the vascularizable passages able to allow host vascular networks to infiltrate the implant article that can facilitate nerve regeneration by enabling exchange of nutrients, oxygen, and/or waste; and
(b) repairing the transected nerve by facilitating growth of nerve fibers from at least one severed end of a transected nerve to at least one severed end of another transected nerve to bridge a gap between the transected nerve.

30. The method of claim 29, wherein the implant article has one or more branches to facilitate repair of one or more transected nerve injuries.

31. The method of claim 29, further comprising determining a size of the transected nerve injuries by magnetic resonance imaging (MRI).

32. The method of claim 29, further comprising, prior to implanting, introducing one or more of cells, growth factors, or nanoparticles into the implant article through the plurality of vascularizable passages.

33. The method of claim 32, wherein the cells are selected from the group consisting of pluripotent stem cells (iPSCs), embryonic stem cells, mesenchymal stem cells, cells differentiated from stem cells, and other supportive cells.

34. The method of claim 32, wherein the growth factors are selected from the group consisting of neurotrophic factor (BDNF), vascular endothelial growth factor (VEGF), basic fibroblast growth factor (bFGF), calpain inhibitor MDL28170, and glial cell-derived neurotrophic factor (GDNF).

35. The method of claim 32, wherein the nanoparticles are selected from the group consisting of boron nitride nanotubes, gold nanorods, carbon nanotubes, and graphene.
